(19) United States
(12) Patent Application Publication
(10) Pub. No.: US 2010/0010050 A1
(43) Pub. Date: Jan. 14, 2010

(54) FORMULATIONS FOR THE CONTROLLED RELEASE OF AGROCHEMICAL ACTIVE AGENTS

(75) Inventors: Karl Reizlein, Monheim (DE); Horst Gruttmann, Leverkusen (DE); Jürgen Thomzik, Langenfield (DE); Joachim Krüger, Monheim (DE)

Correspondence Address: Baker Donelson Bearman, Caldwell & Berkowitz, PC, 555 Eleventh Street, NW, Sixth Floor, Washington, DC, 20004, US

(73) Assignee: Bayer CropScience AG, Monheim (DE)

(21) Appl. No.: 12/438,204

(22) PCT Filed: Aug. 17, 2007
(86) PCT No.: PCT/EP07/07275

§ 371 Date: Apr. 27, 2009

(30) Foreign Application Priority Data
Aug. 23, 2006 (EP) 06017488.5
Sep. 22, 2006 (EP) 06019848.8

(51) Int. Cl. A01N 51/00; A01N 25/00; A01N 47/10; A01N 43/653; A01P 3/00; A61P 7/04

(52) U.S. Cl. 514/341; 514/770; 514/490; 514/383

(57) ABSTRACT

There have been found novel compositions for the controlled release of agrochemical active substances, containing at least one agrochemical active substance, cellulose ester and calcium carbonate.

The present invention relates to novel formulations for the controlled release of agrochemical active substances, to processes for their preparation, and to their use for controlling harmful insects.

A large number of systems for the controlled release of agrochemical or pharmaceutical active substances have already been described in the literature. They are designed for different types and periods of application and are based on different carrier or matrix materials for achieving a controlled release.

Thus, WO 99/55774 discloses systems for the controlled release of—inter alia—agrochemical active substances in which cellulose esters are employed as the carrier material. However, no way of controlling the release is described, in particular not in the type and manner of the present invention.

There have now been found new compositions containing
at least one agrochemical active substance
at least one cellulose ester
calcium carbonate (Ca₂CO₃).

Furthermore, it has been found that the compositions according to the invention can be prepared by mixing
at least one agrochemical active substance
at least one cellulose ester
calcium carbonate (Ca₂CO₃)

and granulating the mixture by melt extrusion.

Furthermore, it has been found that the compositions according to the invention are highly suitable for applying the agrochemical active substances which they contain.

Surprisingly, it has been found that the release of the active substances from the compositions according to the invention containing them can be controlled by the calcium carbonate content. Here, the release rate of the active substance into the environment increases with increasing calcium carbonate content. Moreover, the compositions are easy to handle. Thus, no agglomerations occur as the result of the high glass transition temperature of the carrier materials employed. Moreover, they are biodegradable.

The compositions according to the invention are generally suitable for all agrochemical active substances which are solid at room temperature. Active substances which are preferred in accordance with the invention are active substances from the classes of the insecticides or fungicides which are solid at room temperature. Especially preferred in accordance with the invention are active substances from the classes of the nicotinyls, carbamates, ketoenols or conazole fungicides which are solid at room temperature. Very especially preferred in accordance with the invention are imidacloprid, methiocarb, tebuconazol and spirotetramat. The active substance content in the compositions according to the invention is generally from 0.1 to 20% by weight, preferably from 0.5 to 15% by weight and especially preferably from 1 to 10% by weight.

Preferred in accordance with the invention are cellulose esters, especially preferably cellulose acetate propionate (CAP). CAP with an acetyl content of from 0.3 to 3% by weight and a propionyl content of from 40 to 50% by weight is very especially preferred. Such a CAP is commercially available. The cellulose ester content generally amounts to from 30 to 90% by weight, preferably from 35 to 85% by weight and especially preferably from 40 to 80% by weight in the compositions according to the invention.

Compositions according to the invention furthermore contain calcium carbonate. The calcium carbonate content in the compositions according to the invention is generally from 5 to 50% by weight, preferably 10 to 40% by weight and especially preferably 15 to 30% by weight.

In a preferred embodiment, the compositions according to the invention furthermore contain at least one emulsifier which is solid at room temperature. In a particularly preferred embodiment, the compositions according to the invention contain at least one nonionic emulsifier from the class of the polyoxyethylene/polyoxypropylene block polymers, the alcohol ethoxylates or the polystyrylphenols which is solid at room temperature. In a very particular embodiment, the compositions according to the invention contain a nonionic emulsifier of the formula (I)

HO(CH₂CH₂O)ₓ(CCH₃HCH₂O)_{y}(CH₂CH₂O)_{z}H (I)

in which
x=75,
y=30 and
z=75,

where the values for x, y and z are averages. Such polymers are commercially available. The amount of emulsifier in the compositions according to the invention is generally from 1 to 25% by weight, preferably from 2 to 20% by weight and especially preferably from 3 to 15% by weight.

If appropriate, the compositions according to the invention contain further formulation auxiliaries, for example if appropriate substances from the groups of the emulsifiers, the anionic or nonionic surfactants, the antifoam agents, the preservatives, the antioxidants, the colorants and/or the inert fillers.

Suitable nonionic surfactants or dispersants are all substances of this type which can conventionally be employed in agrochemical products. The following may be mentioned by preference: polyethylene oxide/polypropylene oxide block copolymers, polyethylene glycol ethers of linear alcohols, reaction products of fatty acids with ethylene oxide and/or propylene oxide, furthermore polyvinyl alcohol, polyvinylpyrrolidone, mixed polymers of polyvinyl alcohol and polyvinylpyrrolidone, and copolymers of (meth)acrylic acid and (meth)acrylic esters, furthermore alkyl ethoxylates and alkylaryl ethoxylates which can, if appropriate, be phosphated and, if appropriate, neutralized with bases, examples which may be mentioned being sorbitol ethoxylates; and polyoxyalkylenamine derivatives.

Suitable anionic surfactants are all substances of this type which can conventionally be employed in agrochemical products. Preferred are alkali metal and alkaline earth metal salts of alkylsulphonic acids or alkylarylsulphonic acids.

Another preferred group of anionic surfactants or dispersants are salts of polystyrenesulphonic acids, salts of polyvinylsulphonic acids, salts of naphthalenesulphonic acid/formaldehyde condensates, salts of condensates of naphthalenesulphonic acid, phenolsulphonic acid and formaldehyde, and salts of lignosulphonic acid.

Antifoams which are suitable are all substances which can conventionally be employed for this purpose in agrochemical products. Silicone oils and magnesium stearate are preferred.

Preservatives which are suitable are all substances which can conventionally be employed for this purpose in agrochemical products of this type. Examples which may be mentioned are Preventol® (from Bayer AG) and Proxel®.

Antioxidants which are suitable are all substances which can conventionally be employed for this purpose in agrochemical products. Butylhydroxytoluene is preferred.

Colorants which are suitable are all substances which can conventionally be employed for this purpose in agrochemical products. The following may be mentioned by way of example: titanium dioxide, carbon black, zinc oxide and blue pigments, and also Permanent Red FGR.

Inert fillers which are suitable are all substances which can conventionally be employed for this purpose in agrochemical products. The following are preferred: inorganic particles such as carbonates, silicates and oxides, and also organic substances such as urea/formaldehyde condensates. Examples which may be mentioned are: kaolin, rutile, silicon dioxide, what is known as highly disperse silica, silica gels, and natural and synthetic silicates, furthermore talc.

While being well tolerated by plants, having favourable toxicity to warm-blooded species and being environmentally sound, the compositions according to the invention are suitable for the protection of plants and plant organs, for increasing yields, for improving the quality of the crop and for controlling animal pests, in particular insects, arachnids, helminths, nematodes and molluscs which are found in agriculture, in horticulture, in forests, in gardens and in leisure facilities. They can preferably be employed as plant protection products. They are affective against normally-sensitive and resistant species, and against all or individual developmental stages. The abovementioned pests include:

From the order of the Anoplura (Phthiraptera), for example Damalinia spp., Haematopinus spp., Linognathus spp., Pediculus spp., Trichodectes spp.

From the class of the Arachnida, for example Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici.

From the class of the Bivalva, for example Dreissena spp.

From the order of the Chilopoda, for example Geophilus spp., Scutigera spp.

From the order of the Coleoptera, for example Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnosterna consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogoderma spp., Tychius spp., Xylotrechus spp., Zabrus spp.

From the order of the Collembola, for example Onychiurus armatus.

From the order of the Dermaptera, for example Forficula auricularia.

From the order of the Diplopoda, for example Blaniulus guttulatus.

From the order of the Diptera, for example Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp. Lucilia spp., Musca spp., Nezara spp., Oestrus spp., Oscinella frit, Pegomyia hyoscyami, Phorbia spp., Stomoxys spp., Tabanus spp., Tannia spp., Tipula paludosa, Wohlfahrtia spp.

From the class of the Gastropoda, for example Arion spp., Biomphalaria spp., Bulinus spp., Deroceras spp., Galba spp., Lymnaea spp., Oncomelania spp., Succinea spp.

From the class of the helminths, for example Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp, Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp, Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

Protozoans such as Eimeria can also be controlled.

From the order of the Heteroptera, for example Anasa tristis, Antestiopsis spp., Blissus spp., Calocoris spp., Campylomma livida, Cavelerius spp., Cimex spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Nezara spp., Oebalus spp., Pentomidae, Piesma quadrata, Piezodorus spp., Psallus seriatus, Pseudacysta persea, Rhodnius spp., Sahlbergella singularis, Scotinophora spp., Stephanitis nashi, Tibraca spp., Triatoma spp.

From the order of the Homoptera, for example Acyrthosipon spp., Aeneolamia spp., Agonoscena spp., Aleurodes spp., Aleurolobus barodensis, Aleurothrixus spp., Amrasca spp., Anuraphis cardui, Aonidiella spp., Aphanostigma piri, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Carneocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus ribis, Dalbulus spp., Dialeurodes spp., Diaphorina spp., Diaspis spp., Doralis spp., Drosicha spp., Dysaphis spp., Dysmicoccus spp., Empoasca spp., Eriosoma spp., Erythroneura spp., Euscelis bilobatus, Geococcus coffeae, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva fimbriolata, Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes vaporariorum, Trioza spp., Typhlocyba spp., Unaspis spp., Viteus vitifolii.

From the order of the Hymenoptera, for example Diprion spp., Hoplocampa spp., Lasius spp., Monomorium pharaonis, Vespa spp.

From the order of the Isopoda, for example Armadillidium vulgare, Oniscus asellus, Porcellio scaber.

From the order of the Isoptera, for example Reticulitermes spp., Odontotermes spp.

From the order of the Lepidoptera, for example Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Cheimatobia brumata, Chilo spp., Choristoneura fumiferana, Clysia ambiguella, Cnaphalocerus spp., Earias insulana, Ephestia kuehniella, Euproctis chrysorrhoea, Euxoa spp., Feltia spp., Galleria mellonella, Helicoverpa spp., Heliothis spp., Hofmannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.

From the order of the Orthoptera, for example Acheta domesticus, Blatta orientalis, Blattella germanica, Gryllotalpa spp., Leucophaea maderae, Locusta spp., Melanoplus spp., Periplaneta americana, Schistocerca gregaria.

From the order of the Siphonaptera, for example Ceratophyllus spp., Xenopsylla cheopis.

From the order of the Symphyla, for example Scutigerella immaculata.

From the order of the Thysanoptera, for example Baliothrips biformis, Enneothrips flavens, Frankliniella spp., Heliothrips spp., Hercinothrips femoralis, Kakothrips spp., Rhipiphorothrips cruentatus, Scirtothrips spp., Taeniothrips cardamoni, Thrips spp.

From the order of the Thysanura, for example Lepisma saccharina.

The plant-parasitic nematodes include, for example, Anguina spp., Aphelenchoides spp., Belonoaimus spp., Bursaphelenchus spp., Ditylenchus dipsaci, Globodera spp., Heliocotylenchus spp., Heterodera spp., Longidorus spp., Meloidogyne spp., Pratylenchus spp., Radopholus similis, Rotylenchus spp., Trichodorus spp., Tylenchorhynchus spp., Tylenchulus spp., Tylenchulus semipenetrans, Xiphinema spp.

If the active substance combinations according to the invention contain at least one fungicidal active substance, they are suitable for controlling phytopathogenic fungi such as Plasmodiophoromycetes, Oomycetes, Chytridiomycetes, Zygomycetes, Ascomycetes, Basidiomycetes, Deuteromycetes and the like.

Examples which may be mentioned, but not by limitation, of some pathogens of fungal diseases which come under the abovementioned general terms are:

Diseases caused by powdery mildew pathogens, such as, for example

Blumeria species such as, for example, Blumeria graminis;

Podosphaera species such as, for example, Podosphaera leucotricha;

Sphaerotheca species such as, for example, Sphaerotheca fuliginea;

Uncinula species such as, for example, Uncinula necator;

Diseases caused by rust pathogens such as, for example,

Gymnosporangium species such as, for example, Gymnosporangium sabinae

Hemileia species such as, for example, Hemileia vastatrix;

Phakopsora species such as, for example, Phakopsora pachyrhizi and Phakopsora meibomiae;

Puccinia species such as, for example, Puccinia recondita or Puccinia triticina;

Uromyces species such as, for example, Uromyces appendiculatus;

Diseases caused by pathogens from the Oomycetes group such as, for example,

Bremia species such as, for example, Bremia lactucae;

Peronospora species such as, for example, Peronospora pisi or P. brassicae;

Phytophthora species such as, for example, Phytophthora infestans;

Plasmopara species such as, for example, Plasmopara viticola;

Pseudoperonospora species such as, for example, Pseudoperonospora humuli or Pseudoperonospora cubensis;

Pythium species such as, for example, Pythium ultimum;

Leaf spot diseases and leaf wilts caused by, for example,

Alternaria species such as, for example, Alternaria solani;

Cercospora species such as, for example, Cercospora beticola;

Cladiosporum species such as, for example, Cladiosporium cucumerinum;

Cochliobolus species such as, for example, Cochliobolus sativus (conidial form: Drechslera, syn: Helminthosporium);

Colletotrichum species such as, for example, Colletotrichum lindemuthanium;

Cycloconium species such as, for example, Cycloconium oleaginum;

Diaporthe species such as, for example, Diaporthe citri;

Elsinoe species such as, for example, Elsinoe fawcettii;

Gloeosporium species such as, for example, Gloeosporium laeticolor;

Glomerella species such as, for example, Glomerella cingulata;

Guignardia species such as, for example, Guignardia bidwelli;

Leptosphaeria species such as, for example, Leptosphaeria maculans;

Magnaporthe species such as, for example, Magnaporthe grisea;

Mycosphaerella species such as, for example, Mycosphaerella graminicola;

Phaeosphaeria species such as, for example, Phaeosphaeria nodorum;

Pyrenophora species such as, for example, Pyrenophora teres;

Ramularia species such as, for example, Ramularia collo-cygni;

Rhynchosporium species such as, for example, Rhynchosporium secalis;

Septoria species such as, for example, Septoria apii;

Typhula species such as, for example, Typhula incarnata;

Venturia species such as, for example, Venturia inaequalis;

Root and stem diseases caused by, for example,

Corticium species such as, for example, Corticium graminearum;

Fusarium species such as, for example, Fusarium oxysporum;

Gaeumannomyces species such as, for example, Gaeumannomyces graminis;

Rhizoctonia species such as, for example, Rhizoctonia solani;

Tapesia species such as, for example, Tapesia acuformis;

Thielaviopsis species such as, for example, Thielaviopsis basicola;

Ear and panicle diseases (including maize cobs), caused by, for example,

Alternaria species such as, for example, Alternaria spp.;

Aspergillus species such as, for example, Aspergillus flavus;

Cladosporium species such as, for example, Cladosporium spp.;

Claviceps species such as, for example, Claviceps purpurea;

Fusarium species such as, for example, Fusarium culmorum;

Gibberella species such as, for example, Gibberella zeae;

Monographella species such as, for example, Monographella nivalis;

Diseases caused by smuts such as, for example,

Sphacelotheca species such as, for example, Sphacelotheca reiliana;

Tilletia species such as, for example, Tilletia caries;

Urocystis species such as, for example, Urocystis occulta;

Ustilago species such as, for example, Ustilago nuda;

Fruit rot caused by, for example,

Aspergillus species such as, for example, Aspergillus flavus;

Botrytis species such as, for example, Botrytis cinerea;

Penicillium species such as, for example, Penicillium expansum;

Sclerotinia species such as, for example, Sclerotinia sclerotiorum;

Verticilium species such as, for example, Verticilium alboatrum;

Seed- and soil-borne rots and wilts, and seedling diseases, caused by, for example,

Fusarium species such as, for example, Fusarium culmorum;

Phytophthora species such as, for example, Phytophthora cactorum;

Pythium species such as, for example, Pythium ultimum;

Rhizoctonia species such as, for example, Rhizoctonia solani;

Sclerotium species such as, for example, Sclerotium rolfsii;

Cankers, galls and witches' broom disease, caused by, for example,

Nectria species such as, for example, Nectria galligena;

Wilts caused by, for example,

Monilinia species such as, for example, Monilinia laxa;

Deformations of leaves, flowers and fruits, caused by, for example,

Taphrina species such as, for example, Taphrina deformans;

Degenerative diseases of woody species, caused by, for example,

Esca species such as, for example, Phaemoniella clamydospora;

Diseases of inflorescences and seeds, caused by, for example,

Botrytis species such as, for example, Botrytis cinerea;

Diseases of the plant tubers, caused by, for example,

Rhizoctonia species such as, for example, Rhizoctonia solani;

Diseases caused by bacterial pathogens such as, for example,

Xanthomonas species such as, for example, Xanthomonas campestris pv. oryzae;

Pseudomonas species such as, for example, Pseudomonas syringae pv. lachrymans;

Erwinia species such as, for example, Erwinia amylovora;

The following diseases of soybeans can preferably be controlled:

Fungal diseases on leaves, stems, pods and seeds caused by, for example,

alternaria leaf spot (Alternaria spec. atrans tenuissima), anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi), scab (Sphaceloma glycines), stemphylium leaf blight (Stemphylium botryosum), target spot (Corynespora cassiicola)

Fungal diseases on roots and the stem base caused by, for example,

black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmopspora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

In addition to the agrochemical active substances which have already been mentioned above, the compositions according to the invention may contain further active substances such as insecticides, attractants, sterilants, bactericides, acaricides, nematicides, fungicides, growth-regulating substances, herbicides, safeners, fertilizers or semiochemicals.

Especially advantageous mixing partners are, for example, the following:

Fungicides:

Nucleic Acid Synthesis Inhibitors

benalaxyl, benalaxyl-M, bupirimate, chiralaxyl, clozylacon, dimethirimol, ethirimol, furalaxyl, hymexazol, mefenoxam, metalaxyl, metalaxyl-M, ofurace, oxadixyl, oxolinic acid

Mitosis and Cell Division Inhibitors

benomyl, carbendazim, diethofencarb, ethaboxam, fuberidazole, pencycuron, thiabendazole, thiophanate-methyl, zoxamide

Respiratory Chain Inhibitors Complex I

diflumetorim

Respiratory Chain Inhibitors Complex II

boscalid, carboxin, fenfuram, flutolanil, furametpyr, furmecyclox, mepronil, oxycarboxin, penthiopyrad, thifluzamide

Respiratory Chain Inhibitors Complex III

azoxystrobin, cyazofamid, dimoxystrobin, enestrobin, famoxadone, fenamidone, fluoxastrobin, kresoxim-methyl, metominostrobin, orysastrobin, pyraclostrobin, picoxystrobin, trifloxystrobin

Decouplers

dinocap, fluazinam

ATP Production Inhibitors

fentin acetate, fentin chloride, fentin hydroxide, silthiofam

Amino Acid and Protein Biosynthesis Inhibitors

andoprim, blasticidin-S, cyprodinil, kasugamycin, kasugamycin hydrochloride hydrate, mepanipyrim, pyrimethanil

Signal Transduction Inhibitors

fenpiclonil, fludioxonil, quinoxyfen

Lipid and Membrane Synthesis Inhibitors

chlozolinate, iprodione, procymidone, vinclozolin

ampropylfos, ampropylfos-potassium, edifenphos, etridiazol, iprobenfos (IBP), isoprothiolan, pyrazophos

tolclofos-methyl, biphenyl

iodocarb, propamocarb, propamocarb hydrochloride, propamocarb-fosetylate

Ergosterol Biosynthesis Inhibitors

fenhexamid,

azaconazole, bitertanol, bromuconazole, cyproconazole, diclobutrazole, difenoconazole, diniconazole, diniconazole-M, epoxiconazole, etaconazole, fenarimol, fenbuconazole, fluquinconazole, flurprimidol, flusilazole, flutriafol, furconazole, furconazole-cis, hexaconazole, imazalil, imazalil-sulphate, imibenconazole, ipconazole, metconazole, myclobutanil, nuarimol, oxpoconazole, paclobutrazole, penconazole, pefurazoate, prochloraz, propiconazole, prothioconazole, pyrifenox, simeconazole, tetraconazole, triadimefon, triadimenol, triflumizol, triforine, triticonazole, uniconazole, voriconazole, viniconazole,

aldimorph, dodemorph, dodemorph acetate, fenpropidin, fenpropimorph, spiroxamine, tridemorph,

naftifin, pyributicarb, terbinafin

Cell Wall Synthesis Inhibitors

benthiavalicarb, bialaphos, dimethomorph, flumorph, iprovalicarb, mandipropamid, polyoxins, polyoxorim, validamycin A

Melanin Biosynthesis Inhibitors

capropamid, diclocymet, fenoxanil, phthalide, pyroquilon, tricyclazole

Resistance Induction acibenzolar-S-methyl, probenazole, tiadinil

Multi-Site

captafol, captan, chlorothalonil, copper salts such as copper hydroxide, copper naphthenate, copper oxychloride, copper sulphate, copper oxide, oxine-copper and Bordeaux mixture, dichlofluanid, dithianon, dodine, dodine free base, ferbam, folpet, fluorofolpet, guazatine, guazatine acetate, iminoctadine, iminoctadine albesilate, iminoctadine triacetate, mancopper, mancozeb, maneb, metiram, metiram zinc, propineb, sulphur and sulphur preparations containing calcium polysulphide, thiram, tolylfluanid, zineb, ziram

Other Fungicides

amibromdol, benthiazole, bethoxazin, capsimycin, carvone, quinomethionate, chloropicrin, cufraneb, cyflufenamid, cymoxanil, dazomet, debacarb, diclomezine, dichlorophen, dicloran, difenzoquat, difenzoquat methylsulphate, diphenylamine, ferimzone, flumetover, flusulfamide, fluopicolide, fluoroimide, fosetyl-aluminium, fosetyl-calcium, fosetyl-sodium, hexachlorobenzene, 8-hydroxyquinoline sulphate, irumamycin, methasulphocarb, metrafenon, methyl isothiocyanate, mildiomycin, natamycin, nickel dimethyldithiocarbamate, nitrothal-isopropyl, octhilinone, oxamocarb, oxyfenthiin, pentachlorophenol and salts, 2-phenylphenol and salts, piperalin, propanosine-sodium, proquinazid, pyribencarb, pyrrolnitrine, quintozene, tecloftalam, tecnazene, triazoxide, trichlamide, valiphenal, zarilamide,

N-methyl-2-(2-{[6-(3-chloro-2-methylphenoxy)-5-fluoropyrimidin-4-yl]oxy}phenyl)-2-(methoxy-imino)acetamide,

N-methyl-2-[[[[1-[3-(1-fluoro-2-phenylethyl)oxy]phenyl]ethylidene]amino]oxy]methyl]-alpha-(methoxyimino)-alphaE-benzacetamide,

cis-1-(4-chlorophenyl)-2-(1H-1,2,4-triazol-1-yl)-cycloheptanol,

1-[(4-methoxyphenoxy)methyl]-2,2-dimethylpropyl-1H-imidazole-1-carboxylic acid,

2,3,5,6-tetrachloro-4-(methylsulphonyl)pyridine,

2-butoxy-6-iodo-3-propylbenzopyranon-4-one,

2-chloro-N-(2,3-dihydro-1,1,3-trimethyl-1H-inden-4-yl)-3-pyridinecarboxamide,

3,4,5-trichloro-2,6-pyridinedicarbonitrile,

3,4-dichloro-N-(2-cyanophenyl)isothiazole-5-carboxamide(Isotianil)

3-[5-(4-chlorophenyl)-2,3-dimethylisoxazolidin-3-yl]pyridine,

5-chloro-6-(2,4,6-trifluorophenyl)-N-[(1R)-1,2,2-trimethylpropyl][1,2,4]triazolo[1,5-a]pyrimidin-7-amine,

5-chloro-7-(4-methylpiperidin-1-yl)-6-(2,4,6-trifluorophenyl)[1,2,4]triazolo[1,5-a]pyrimidine,

5-chloro-N-[(1R)-1,2-dimethylpropyl]-6-(2,4,6-trifluorophenyl)-[1,2,4]triazolo[1,5-a]pyrimidin-7-amine,

methyl 2-[[[cyclopropyl[(4-methoxyphenyl)imino]methyl]thio]methyl]-alpha-(methoxymethylene)-benzoacetate,

methyl 1-(2,3-dihydro-2,2-dimethyl-1H-inden-1-yl)-1H-imidazole-5-carboxylate,

N-(3′,4′-dichloro-5-fluorobiphenyl-2-yl)-3-(difluoromethyl)-1-methyl-1H-pyrazole-4-carboxamide,

N-(3-ethyl-3,5,5-trimethyl-cyclohexyl)-3-formylamino-2-hydroxybenzamide,

N-(4-chloro-2-nitrophenyl)-N-ethyl-4-methylbenzenesulphonamide,

N-(4-chlorobenzyl)-3-[3-methoxy-4-(prop-2-yn-1-yloxy)phenyl]propanamide,

N-[(4-chlorophenyl)(cyano)methyl]-3-[3-methoxy-4-(prop-2-yn-1-yloxy)phenyl]propanamide,

N-(5-bromo-3-chloropyridin-2-yl)methyl-2,4-dichloronicotinamide,

N-[1-(5-bromo-3-chloropyridin-2-yl)ethyl]-2,4-dichloronicotinamide,

(2S)-N-[2-[4-[[3-(4-chlorophenyl)-2-propinyl]oxy]-3-methoxyphenyl]ethyl]-3-methyl-2-[(methyl-sulphonyl)amino]butanamide,

N-{(Z)-[(cyclopropylmethoxy)imino][6-(difluoromethoxy)-2,3-difluorophenyl]methyl}-2-benzacetamide,

N-{2-[1,1′-bi(cyclopropyl)-2-yl]phenyl}-3-(difluoromethyl)-1-methyl-1H-pyrazole-4-carboxamide,

N-{2-[3-chloro-5-(trifluoromethyl)pyridin-2-yl]ethyl}-2-(trifluoromethyl)benzamide,

N-ethyl-N-methyl-N′-{2-methyl-5-(trifluoromethyl)-4-[3-(trimethylsilyl)propoxy]phenyl}imido-formamide,

O-[1-[(4-methoxyphenoxy)methyl]-2,2-dimethylpropyl]-1H-imidazole-1-carbothioic acid,

2-amino-4-methyl-N-phenyl-5-thiazolecarboxamide,

2,4-dihydro-5-methoxy-2-methyl-4-[[[[1-[3-(trifluoromethyl)phenyl]ethylidene]amino]oxy]methyl]-phenyl]-3H-1,2,4-triazol-3-one (CAS No. 185336-79-2),

N-(6-methoxy-3-pyridinyl)cyclopropanecarboxamide,

Bactericides:

Bronopol, dichlorophen, nitrapyrin, nickel dimethyl dithiocarbamate, kasugamycin, octhilinone, furancarboxylic acid, oxytetracyclin, probenazole, streptomycin, tecloftalam, copper sulphate and other copper preparations.

Insecticides/Acaricides/Nematicides:

Acetylcholine Esterase (AChE) Inhibitors

Carbamates, for example alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulfan, chloethocarb, dimetilan, ethiofencarb, fenobucarb, fenothiocarb, formetanate, furathiocarb, isoprocarb, metam-sodium, methomyl, metolcarb, oxamyl, pirimicarb, promecarb, propoxur, thiodicarb, thiofanox, trimethacarb, XMC, xylylcarb, triazamate

Organophosphates, for example acephate, azamethiphos, azinphos (-methyl, -ethyl), bromophosethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-S-methyl, demeton-S-methylsulphon, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion

Sodium Channel Modulators/Voltage-Dependent Sodium Channel Blockers

Pyrethroids, for example acrinathrin, allethrin (d-cis-trans, d-trans), beta-cyfluthrin, bifenthrin, bioallethrin, bioallethrin-S-cyclopentyl isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, deltamethrin, empenthrin (1R isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R trans-isomer), prallethrin, profluthrin, protrifenbute, pyresmethrin, resmethrin, RU 15525, silafluofen, tau-fluvalinate, tefluthrin, terallethrin, tetramethrin (1R isomer), tralomethrin, transfluthrin, ZXI 8901, pyrethrins (pyrethrum)

DDT

Oxadiazines, for example indoxacarb

Semicarbazones, for example metaflumizon (BAS 3201)

Acetylcholine Receptor Agonists/Antagonists

Chloronicotinyls, for example acetamiprid, clothianidin, dinotefuran, nitenpyram, nithiazine, thiacloprid, thiamethoxam

Nicotine, bensultap, cartap

Acetylcholine Receptor Modulators

Spinosyns, for example spinosad

GABA-controlled chloride channel antagonists

Organochlorines, for example camphechlor, chlordane, endosulfan, gamma-HCH, HCH, heptachlor, lindane, methoxychlor

Fiproles, for example acetoprole, ethiprole, fipronil, pyrafluprole, pyriprole, vaniliprole

Chloride Channel Activators

Mectins, for example abamectin, emamectin, emamectin benzoate, ivermectin, lepimectin, milbemycin

Juvenile hormone mimetics, for example diofenolan, epofenonane, fenoxycarb, hydroprene, kinoprene, methoprene, pyriproxifen, triprene

Ecdysone Agonists/Disruptors

Diacylhydrazines, for example chromafenozide, halofenozide, methoxyfenozide, tebufenozide

Chitin Biosynthesis Inhibitors

Benzoylureas, for example bistrifluron, chlofluazuron, diflubenzuron, fluazuron, flucycloxuron, flufenoxuron, hexaflumuron, lufenuron, novaluron, noviflumuron, penfluron, teflubenzuron, triflumuron

buprofezin

cyromazine

Inhibitors of Ixidative Phosphorylation, ATP Disruptors

diafenthiuron

Organotin compounds, for example azocyclotin, cyhexatin, fenbutatin oxide

Decouplers of oxidative phosphorylation by interrupting the H-proton gradient

Pyrroles, for example chlorfenapyr

Dinitrophenols, for example binapacyrl, dinobuton, dinocap, DNOC, meptyldinocap

Site-I electron transport inhibitors

METIs, for example fenazaquin, fenpyroximate, pyrimidifen, pyridaben, tebufenpyrad, tolfenpyrad

hydramethylnon

dicofol

Site-II Electron Transport Inhibitors

rotenone

Site-III Electron Transport Inhibitors

acequinocyl, fluacrypyrim

Microbial disruptors of the insect gut membrane

Bacillus thuringiensis strains

Fat biosynthesis inhibitors

Tetronic acids,

for example spirodiclofen, spiromesifen

Tetramic acids,

for example cis-3-(2,5-dimethylphenyl)-4-hydroxy-8-methoxy-1-azaspiro[4.5]dec-3-en-2-one

Carboxamides

for example flonicamid

Octopaminergic Agonists

for example amitraz

Inhibitors of magnesium-stimulated ATPase,

propargite

Nereistoxin Analogues

for example thiocyclam hydrogen oxalate, thiosultap sodium

Ryanodin receptor agonists,

Benzoic dicarboxamides,

for example flubendiamide

Anthranilamides,

for example rynaxypyr(3-bromo-N-{4-chloro-2-methyl-6[(methylamino)carbonyl]phenyl}-1-(3-chloropyridin-2-yl)-1H-pyrazole-5-carboxamide)

Biologicals, Hormones or Pheromones

azadirachtin, Bacillus spec., Beauveria spec., codlemone, Metarrhizium spec., Paecilomyces spec., thuringiensin, Verticillium spec.

Active Substances with Unknown or Unspecific Mechanisms of Action

Fumigants, for example aluminium phosphide, methyl bromide, sulfuryl fluoride

Antifeedants, for example cryolite, flonicamid, pymetrozine

Mite growth inhibitors, for example clofentezine, etoxazole, hexythiazox

Amidoflumet, benclothiaz, benzoximate, bifenazate, bromopropylate, buprofezin, quinomethionate, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnon, japonilure, metoxadiazone, petroleum, piperonyl butoxide, potassium oleate, pyridalyl, sulfluramid, tetradifon, tetrasul, triarathene, verbutin,

A mixture of substances for improving the plant's properties is also possible.

When employed in their commercially available formulations and in the use forms prepared with these formulations, the compositions according to the invention may furthermore be present as a mixture with synergists. Synergists are compounds by means of which the activity of the active substances present in the compositions according to the invention is increased without being necessary for the added synergist to be active itself.

When employed in their commercially available formulations and in the use forms prepared with these formulations, the compositions according to the invention may furthermore be present as a mixture with inhibitors which reduce the degradation of the agrochemical active substance present after it has been applied to the environment of the plant, to the surface of plant parts or in plant tissues.

The active substance content of the use forms prepared from the compositions according to the invention can vary within wide limits. The active substance concentration of the use forms can be from 0.00000001 up to 95% by weight of active substance, preferably between 0.00001 and 20% by weight.

The application is accomplished in a customary manner adapted to suit the use forms.

All plants can be treated in accordance with the invention. In a preferred embodiment, plant species and plant varieties which are found in the wild or are obtained by traditional biological breeding methods, such as hybridization or protoplast fusion, are treated. In a further preferred embodiment, transgenic plants and plant varieties which have been obtained by recombinant methods, if appropriate in combination with traditional methods (genetically modified organisms) are treated.

Particularly preferably, plants of the plant cultivars which are in each case commercially available or in use are treated according to the invention. Plant cultivars are understood as meaning plants with new properties (“traits”) which have been obtained by conventional cultivation, by mutagenesis or else by recombinant DNA techniques. These may be cultivars, biotypes or genotypes.

Depending on the plant species or plant cultivars, their location and growth conditions (soils, climate, vegetation period, nutrition), the treatment according to the invention may also result in superadditive (“synergistic”) effects. Thus, for example, reduced application rates and/or extensions of the activity spectrum and/or an increase in the activity of the substances and compositions that can be used according to the invention, better plant growth, increased tolerance to high or low temperatures, increased tolerance to drought or to water or soil salinity, increased flowering performance, easier harvesting, accelerated maturation, higher yields, better quality and/or a higher nutritional value of the harvested products, better storage ability and/or processability of the harvested products which exceed the effects which were actually to be expected are possible.

The preferred transgenic plants or plant cultivars (i.e. those obtained by genetic engineering) which are to be treated according to the invention include all plants which, as a result of the recombinant modification, received genetic material which imparted particularly advantageous useful properties (“traits”) to these plants. Examples of such properties are better plant growth, increased tolerance to high or low temperatures, increased tolerance to drought or to water or soil salinity, increased flowering performance, easier harvesting, accelerated maturation, higher yields, better quality and/or a higher nutritional value of the harvested products, better storage ability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active substances. Examples of transgenic plants which may be mentioned are the important crop plants, such as cereals (wheat, rice), maize, soya beans, potatoes, sugar beet, tomatoes, peas and other vegetables, cotton, tobacco, oilseed rape and also fruit plants (with the fruits apples, pears, citrus fruits and grapes), and particular emphasis is given to maize, soya beans, potatoes, cotton, tobacco and oilseed rape. Traits that are emphasized in particular are increased defence of the plants against insects, arachnids, nematodes, slugs and snails as a result of toxins formed in the plants, in particular those formed in the plants by the genetic material from Bacillus Thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinbelow referred to as “Bt plants”). Traits which are also particularly emphasized are the increased defence of plants against fungi, bacteria and viruses by systemic acquired resistance (SAR), systemin, phytoalexins, elicitors and resistance genes and the correspondingly expressed proteins and toxins. Traits that are furthermore particularly emphasized are the increased tolerance of the plants to certain herbicidally active substances, for example imidazolinones, sulphonylureas, glyphosate or phosphinothricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosates, for example maize, cotton, soya beans), Liberty Link® (tolerance to phosphinothricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned also include the varieties sold under the name Clearfield® (for example maize). Of course, these statements also apply to plant varieties which will be developed, or reach the market, in the future and which have these genetic traits or have genetic traits yet to be developed.

The plants which have been detailed can particularly advantageously be treated according to the invention with the compositions according to the invention. The preferred ranges stated hereinabove for the compositions also apply to the treatment of these plants. The treatment of plants with the compositions which are specifically mentioned in the present text may be particularly emphasized.

The examples which follow illustrate the invention without imposing any limitation.

### EXAMPLES

### Preparation Examples

The following compositions according to the invention were prepared:

| | Example 1 | Example 2 | Example 3 | Example 4 |
|---|---|---|---|---|
| Active substance | Imida- | Imida- | Imida- | Imida- |
| | cloprid | cloprid | cloprid | cloprid |
| Active substance/% | 5 | 5 | 5 | 5 |
| by weight | | | | |
| Cellulose ester | CAP | CAP | CAP | CAP |
| Cellulose ester/% | 70 | 55 | 75 | 60 |
| by weight | | | | |
| CaCO₃/% | 15 | 30 | 15 | 30 |
| by weight | | | | |
| Emulsifier (I)/% | 10 | 10 | 5 | 5 |
| by weight | | | | |

Release of the Active Substance

The release of the active substance was determined for the exemplary formulation after introducing the formulation into water:

Release of active substance in %

| | After 48 h | After 168 h |
|---|---|---|
| Example 1 | 18 | 26 |
| Example 2 | 29 | 38 |
| Example 3 | 7 | 13 |
| Example 4 | 12 | 20 |

1. A composition comprising
at least one agrochemical active substances
at least one cellulose ester and
calcium carbonate (Ca₂CO₃).

2. A composition according to claim 1 additionally comprising at least one emulsifier which is solid at room temperature.

3. A composition according to claim 1, comprising as said agrochemical active substance, imidacloprid, methiocarb, tebuconazole and/or spirotetramat.

4. A composition according to claim 1, comprising as said agrochemical active substances imidacloprid.

5. A composition according to claim 1 comprising
0.1 to 20% by weight of said agrochemical active substance
30 to 90% by weight of said cellulose ester
5 to 50% by weight of said calcium carbonate.

6. A composition according to claim 5, comprising 1 to 25% by weight of an emulsifier which is solid at room temperature.

7. A method of controlling harmful insects and/or phytopathogenic fungi, comprising applying a composition according to one or more of claim 1.

8. A composition according to claim 1 which is suitable for controlling harmful insects and/or phytopathogenic fungi.
